(19) United States
(12) Patent Application Publication
(10) Pub. No.: US 2024/0027478 A1
(43) Pub. Date: Jan. 25, 2024

(54) TREATMENT OF HEMOPHILIA WITH FITUSIRAN

(71) Applicant: Genzyme Corporation, Cambridge, MA (US)

(72) Inventors: Stacey Poloskey, Sudbury, MA (US); Shauna Andersson, Lexington, MA (US); Pratik R. Bhagunde, New Providence, NJ (US); Yue Cui, Lexington, MA (US); Sajida Iqbal, Natick, MA (US); Baisong Mei, Waban, MA (US)

(73) Assignee: Genzyme Corporation, Cambridge, MA (US)

(21) Appl. No.: 18/256,199

(22) PCT Filed: Dec. 6, 2021
(86) PCT No.: PCT/US2021/062078

§ 371 Date: Jun. 6, 2023

Related U.S. Application Data
(60) Provisional application No. 63/275,344, filed on Nov. 3, 2021.
(60) Provisional application No. 63/272,629, filed on Oct. 27, 2021.
(60) Provisional application No. 63/121,973, filed on Dec. 6, 2020.

(51) Int. Cl. G01N 33/86; A61K 31/713; A61P 7/04

(52) U.S. Cl. CPC G01N 33/86; A61K 31/713; A61P 7/04; G01N 2333/8128; G01N 2800/224; G01N 2800/52

(57) ABSTRACT

The present disclosure provides a method that aims to maintain a favorable benefit-risk balance for patients with hemophilia A or B with or without inhibitors treated with fitusiran.

### CROSS-REFERENCE TO RELATED APPLICATIONS

The present application claims priority from U.S. Applications 63/121,973, filed Dec. 6, 2020; 63/272,629, filed Oct. 27, 2021; and 63/275,344, filed Nov. 3, 2021. The contents of the priority applications are incorporated by reference herein in their entirety.

### SEQUENCE LISTING

The instant application contains a Sequence Listing which has been submitted electronically in ASCII format and is hereby incorporated by reference in its entirety. Said ASCII copy, created on Dec. 2, 2021, is named 022548_WO086_SL.txt and is 4,656 bytes in size.

### BACKGROUND OF THE INVENTION

Maintenance of normal hemostasis relies on a regulated set of simultaneously occurring procoagulant and anticoagulant processes, in which thrombin plays a central role. Hemophilia A and B are inherited bleeding disorders caused by deficiencies in factors VIII and IX, respectively. Bleeding in hemophilia A and B arises from insufficient thrombin generation (Peyvandi et al., Lancet (2016) 388(10040):187-97). Without effective treatment, patients with hemophilia experience recurrent bleeding, which lead to major disability due to chronic hemarthropathy and significant pain, and can be life-threatening (Pipe et al., Haemophilia (2007) 13 Suppl 4:1-16).

Significant unmet needs and management challenges continue to exist for all hemophilia populations despite treatment advances. While prophylaxis based on replacement therapy with factor VIII or IX is considered the cornerstone of hemophilia management, it has significant limitations. For example, injections of factor replacement for prophylaxis are burdensome and impractical, often requiring multiple intravenous infusions per week (Peyvandi, supra; Ljung and Andersson, Br J Haematol. (2015) 169(6):777-86; Srivastava et al., Haemophilia (2013) 19(1):e1-47; Bauer, Am J Manag Care (2015) 21(6 Suppl):S112-22; Mannucci and Franchini, Blood Transfus. (2013) 11(Suppl 4):s77-81). Factor replacement is also limited by difficulties with venous access and risk of infections (Balkaransingh and Young, Ther Adv Hematol. (2018) 9(2):49-61; Valentino et al., Blood Rev. (2011) 25(1):11-5). Limitations in delivering factor replacement also result in a large proportion of the world's hemophilia population without access in the first instance to prophylaxis treatment (Hemophilia, W.F.O. Treatment Safety and Supply. 2020).

Additionally, treatment with factor replacement products can result in the development of inhibitory alloantibodies, rendering the factor treatment ineffective (Morfini et al., Haemophilia (2007) 13(5):606-12). These inhibitors, which typically occur in childhood, limit treatment options and dramatically worsen the prognosis of hemophilia. Moreover, those with persistent inhibitors typically have a lower quality of life, greater joint disease, greater surgical risk, and higher mortality, including a higher risk of death from hemophilia-related bleeding complications, when compared to patients without inhibitors (Morfini, supra; Oladapo et al., Orphanet J Rare Dis. (2018) 13(1):198). Current treatment strategies for individuals with persistent inhibitors include immune tolerance induction (ITI) and prophylaxis with bypassing agents (BPAs) such as activated prothrombin complex concentrates (aPCC) and recombinant activated factor VII (rFVIIa) (Benson et al., Eur J Haematol. (2012) 88(5):371-79; Collins et al., Br J Haematol. (2013) 160(2):153-70; Kempton et al., Blood (2014) 124(23):3365-72; Astermark et al., Haemophilia (2007) 13(1):38-45; Eichinger et al., Eur J Clin Invest. (2009) 39(8):707-13).

Thus, there remains a need for alternative treatments for subjects having a bleeding disorder, such as hemophilia.

### SUMMARY OF THE INVENTION

The present disclosure provides methods of prophylactically treating hemophilia A or hemophilia B with fitusiran to prevent or reduce the frequency of bleeding episodes in patients, optionally in patients who are 12 years of age or older, and provides fitusiran for use in these methods. In one aspect, the present disclosure provides a method of prophylactic treatment of a patient with hemophilia A or B with or without inhibitors, or a method of reducing thrombotic risk in a patient receiving fitusiran for prophylactic treatment of hemophilia A or B with or without inhibitors, and fitusiran for use in such a method.

In some embodiments, the method comprises: a) subcutaneously administering to the patient in need thereof 50 mg of fitusiran every two months or every eight weeks, b) obtaining a measurement of an antithrombin (AT) level in the patient, and c) subcutaneously administering to the patient 80 mg of fitusiran every two months or every eight weeks, or 50 mg of fitusiran every month or every four weeks, if the AT level is >X %, wherein X=25 or 35.

In some embodiments, the method further comprises d) continuing step a) if the AT level is Y-X %, wherein Y=10 or 15, and/or e) discontinuing fitusiran treatment if the AT level is <Y %, wherein Y=10 or 15. In further embodiments, step b) is performed at least twice and step c) is performed only when both measured AT levels are >X %, or step e) is performed only when both measured AT levels are <Y %. In some embodiments, X is 35 and Y is 15.

In some embodiments, the method further comprises, after performing step c): i) obtaining a measurement of AT level in the patient; and ii) subcutaneously administering to the patient 80 mg of fitusiran every month or every four weeks, if the AT level of step i) is >25% or >35%, or iii) otherwise continuing step c). In further embodiments, step i) is performed at least twice and step ii) is performed only when both measured AT levels of step i) are >25% or >35%.

In some embodiments, the method further comprises, after performing step c) wherein step c) comprises subcutaneously administering to the patient 80 mg of fitusiran every two months or every eight weeks: i) obtaining a measurement of AT level in the patient; and ii) subcutaneously administering to the patient 50 mg of fitusiran every month or every four weeks, if the AT level of step i) is >25% or >35%, or iii) otherwise continuing step c). In further embodiments, step i) is performed at least twice and step ii) is performed only when both measured AT levels of step ii) are >25% or >35%. In certain embodiments, the method further comprises, after performing step ii): A) obtaining a measurement of AT level in the patient; and B) subcutaneously administering to the patient 80 mg of fitusiran every month or every four weeks, if the AT level of step A) is >25% or >35%, or C) otherwise continuing step ii). In further embodiments, step A) is performed at least twice and step B) is performed only when both measured AT levels are >25% or >35%.

In some embodiments of the present disclosure, X is 35% and Y is 15%. In other embodiments, X is 35% and Y is 10%. In other embodiments, X is 25% and Y is 15%. In other embodiments, X is 25% and Y is 10%.

In one aspect, the present disclosure provides a method of prophylactic treatment of a patient with hemophilia A or B with or without inhibitors, or reducing thrombotic risk in a patient receiving fitusiran for prophylactic treatment of hemophilia A or B with or without inhibitors, optionally wherein the patient is twelve years of age or older, and fitusiran for use in such a method, wherein the method comprises: (a) subcutaneously administering to the patient in need thereof fitusiran at a starting dose amount (e.g., selected from 10 mg to 50 mg) at a selected dosing frequency (e.g., every two months (Q2M) or every eight weeks (Q8W)); (b) obtaining a measurement of an antithrombin (AT) level in the patient; and (c) performing one of the following steps: (i) if the AT level is 15-35%, repeating step (a), (ii) if the AT level is >35%, subcutaneously administering to the patient fitusiran at a higher dose amount at the selected dosing frequency, or at the starting dose amount at a higher dosing frequency (e.g., every month (QM) or every four weeks (Q4W)), or (iii) if the AT level is <15%, discontinuing or pausing fitusiran treatment.

In some embodiments, the starting dose amount is 20 mg and the selected dosing frequency is Q2M or Q8W.

In some embodiments, the starting dose amount is 50 mg and the selected dosing frequency is Q2M or Q8W, optionally wherein step (c)(ii) is performed if two measurements of the AT level are >35%, or step (c)(iii) is performed if more than one measurement, optionally two measurements, of the AT level is <15%. In further embodiments, step (c)(ii) comprises subcutaneously administering to the patient fitusiran at 50 mg QM. In certain embodiments, the method comprises, after step (c)(ii), subcutaneously administering to the patient fitusiran at 80 mg QM if the AT level in the patient after step (c)(ii) is >35%, optionally according to two measurements.

In some embodiments, the present method further comprises, after step (c)(iii): (A) subcutaneously administering fitusiran to the patient at 20 mg Q2M; (B) (1) if the AT level of the patient after step (A) is <15%, optionally according to more than measurement, discontinuing fitusiran treatment; (2) if the AT level after step (A) is 15-35%, repeating step (A); or (3) if the AT level after step (A) is >35%, optionally according to two measurements, subcutaneously administering to the patient fitusiran at 20 mg QM.

In some embodiments, the present method comprises subcutaneously administering fitusiran to the patient at a dose amount and a dosing frequency sufficient to maintain the patient's AT level at 15-35%, wherein the administration regimen is optionally selected from 10 mg QM or Q4W, 20 mg QM or Q4W, 20 mg Q2M or Q8W, 50 mg QM or Q4W, 50 mg Q2M or Q8W, or 80 mg QM or Q4W.

In some embodiments, the AT level measurements are obtained during steady state of AT activity, e.g., after the patient has received at least two doses of fitusiran in the prior step. In some embodiments, each AT level measurement is obtained after the patient has received at least two doses of fitusiran at a given dose amount. In some embodiments, the method comprises obtaining a measurement of AT level in the patient every four weeks, every eight weeks, every month, every two months, every four months, every six months, or every 12 months.

In one aspect, the present disclosure provides a method of prophylactic treatment of a patient with hemophilia A or B with or without inhibitors and fitusiran for use in such a method, optionally wherein the patient is twelve years of age or older, wherein the method comprises subcutaneously administering to the patient in need thereof 50 or 80 mg of fitusiran Q2M or Q8W (e.g., 50 mg Q2M or Q8W, or 80 mg Q2M or Q8W).

In another embodiment, the present disclosure provides a method of prophylactic treatment of a patient with hemophilia A or B with or without inhibitors and fitusiran for use in such a method, optionally wherein the patient is twelve years of age or older, wherein the method comprises subcutaneously administering to the patient in need thereof 20 mg of fitusiran QM or Q4W, or Q2M or Q8W.

In another embodiment, the present disclosure provides a method of prophylactic treatment of a patient with hemophilia A or B with or without inhibitors and fitusiran for use in such a method, optionally wherein the patient is twelve years of age or older, wherein the method comprises subcutaneously administering to the patient in need thereof 10 mg of fitusiran QM or Q4W.

In some embodiments, fitusiran is a sodium salt and may be provided in a pharmaceutically acceptable aqueous solution, e.g., a phosphate-buffered saline (PBS), at 50-200 mg/mL, optionally 100 mg/mL, and optionally wherein the PBS has a pH of 7. As used herein, fitusiran weight refers to the weight of fitusiran in a free acid form.

In one aspect, the present disclosure provides an article of manufacture (e.g., a kit that optionally includes instructions for use) for use in the present treatment method. In some embodiments, the article of manufacture is a container containing one or more doses of fitusiran, each dose being 80 mg, 50 mg, 20 mg, or 10 mg, optionally wherein the 80 mg of fitusiran is in 0.8 mL of a phosphate-buffered saline (PBS), the 50 mg of fitusiran is in 0.5 mL of a PBS, the 20 mg of fitusiran is in 0.2 mL of a PBS, or the 10 mg of fitusiran is in 0.1 mL of a PBS, and optionally wherein the PBS has a pH of 7. The container may be, for example, a single-use, single-dose prefilled syringe.

In another aspect, the present disclosure provides fitusiran for the manufacture of a medicament to treat hemophilia A or B with or without inhibitors in the treatment method herein.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 shows the expanded structural formula, chemical formula, and molecular mass of fitusiran. This figure discloses SEQ ID NOs:1 and 2, respectively, in order of appearance.

FIG. 2 is a graph depicting the AT activity of five patients with thrombotic events superimposed on AT activity of Phase 3 patients. The background black circles are AT activity data from only Phase 3 studies. Each line represents an individual patient.

FIG. 3 is a graph depicting the distribution of coefficient of variation (% CV) for intra-individual variability in AT activity at steady state.

FIG. 4 is graph depicting the threshold of average AT for the 95% chance that the next AT value is >10%.

FIG. 5 is a jitter box plot of thrombin generation peak height in Phase I study.

FIG. 6 is a bar graph depicting ABR estimates by AT quartiles in Phase I study.

FIG. 7 is a flow chart depicting a fitusiran escalation scheme. “Q2M”: every two months. “QM”: every month.

FIG. 8 is a flow chart depicting an alternative fitusiran escalation scheme.

FIG. 9 is a flow chart depicting an alternative fitusiran escalation scheme.

FIG. 10 is a schematic depicting the pharmacokinetic/pharmacodynamic (PK/PD) model used to simulate the dynamics of plasma AT activity for patients treated with fitusiran. K_{A}: absorption rate; F: bioavailability with dose effect on bioavailability; V₂: liver central compartment volume; V₃ liver peripheral compartment volume; CL: clearance from liver central compartment; CL₂: liver intercompartmental clearance; Q: clearance into RISC compartment; RV: RISC volume; CL_{R}: clearance out of RISC compartment; Kᵢₙ: AT production rate; Kₒᵤₜ: AT elimination rate; Iₘₐₓ: maximum inhibition of AT production; IC₅₀: RISC concentration for 50% of maximum inhibition of AT production.

FIG. 11 is a flow chart showing the risk mitigation strategy for vascular thrombotic events. “AT”: antithrombin activity; “QM”: once monthly; “Q2M”: every second month; “ss”: steady state. After discontinuing fitusiran due to AT levels<15% at a dose of 50 mg Q2M, patients are eligible to resume treatment with 20 mg Q2M fitusiran if their AT level is ≥22%.

### DETAILED DESCRIPTION OF THE INVENTION

The present disclosure provides a method that aims to maintain a favorable benefit-risk balance for patients with hemophilia A or B with or without inhibitors who are treated with fitusiran. Fitusiran is intended for routine prophylaxis to prevent or reduce the frequency of bleeding episodes in patients, for example adult and adolescent patients (≥12 years old), with hemophilia A or B, including patients with inhibitory antibodies (inhibitors). The present treatment methods carefully calibrate the therapy based on the patient's AT levels so as to minimize the risk of vascular thrombotic events that may result from inappropriately low AT levels (e.g., AT levels<10%).

A hemophilia A or B patient with inhibitors refers to a patient who has developed alloantibodies to the factor he/she has previously received (e.g., factor VIII for hemophilia A patients or factor IX for hemophilia B patients). A hemophilia A or B patient with inhibitors may become refractory to replacement coagulation factor therapies. A patient without inhibitors refers to a patient who does not have such alloantibodies. The present treatment methods may be beneficial for hemophilia A patients with or without inhibitors, as well as for hemophilia B patients with or without inhibitors. As used herein, “hemophilia A or B with or without inhibitors” refers to hemophilia A with or without inhibitors, or hemophilia B with or without inhibitors. As used herein, a patient refers to a human patient, optionally an adolescent or adult human patient that is 12 years of age or older.

### I. FITUSIRAN PHARMACEUTICAL COMPOSITIONS

Hemophilia results in a profound defect in thrombin generation, and further, hemophilia severity is correlated with the inability to generate thrombin. Without being bound by theory, it is believed that fitusiran-mediated lowering of antithrombin (AT) levels will increase thrombin generation and thus improve hemostasis in patients with hemophilia.

The structure of fitusiran is provided herein. Fitusiran is a synthetically, chemically modified double-stranded small interfering RNA (siRNA) oligonucleotide covalently linked to a tri-antennary N-acetyl-galactosamine (GalNAc) ligand targeting the AT3 mRNA in the liver, thereby suppressing the synthesis of antithrombin. See, e.g., Pasi, supra. Antithrombin is encoded by the SERPINC1 gene. The nucleosides in each strand of fitusiran are connected through either 3′-5′ phosphodiester or phosphorothioate linkages, thus forming the sugar-phosphate backbone of the oligonucleotide.

While the fitusiran dosage weight described herein refers to the weight of fitusiran free acid (active moiety), administration of fitusiran to patients herein refers to administration of fitusiran sodium (drug substance) provided in a pharmaceutically suitable aqueous solution (e.g., a phosphate-buffered saline at a physiological pH).

The sense strand and the antisense strand of fitusiran contain 21 and 23 nucleotides, respectively. The 3′ end of the sense strand is conjugated to the GalNAc containing moiety (referred to as L96) through a phosphodiester linkage. The sense strand contains two consecutive phosphorothioate linkages at its 5′ end. The antisense strand contains four phosphorothioate linkages, two at the 3′ end and two at the 5′ end. The 21 nucleotides of the sense strand hybridize with the complementary 21 nucleotides of the antisense strand, thus forming 21 nucleotide base pairs and a two-base overhang at the 3′-end of the antisense strand. See also U.S. Pat. No. 9,127,274, US20170159053, and WO 2019/014187.

The two nucleotide strands of fitusiran are shown below:

sense strand: 5′Gf-ps-Gm-ps-Uf-Um-Af-Am-Cf-Am-Cf-Cf-Af-Um-Uf-Um-Af-Cm-Uf-Um-Cf-Am-Af-L96 3′ (SEQ ID NO:1), and
antisense strand: 5′ Um-ps-Uf-ps-Gm-Af-Am-Gf-Um-Af-Am-Af-Um-Gm-Gm-Uf-Gm-Uf-Um-Af-Am-Cf-Cm-ps-Am-ps-Gm 3′ (SEQ ID NO:2),
wherein

Af=2′-fluoroadenosine (i.e., 2′-deoxy-2′-fluoroadenosine)

Cf=2′-fluorocytidine (i.e., 2′-deoxy-2′-fluorocytidine)

Gf=2′-fluoroguanosine (i.e., 2′-deoxy-2′-fluoroguanosine)

Uf=2′-fluorouridine (i.e., 2′-deoxy-2′-fluorouridine)

Am=2′-O-methyladenosine

Cm=2′-O-methylcytidine

Gm=2′-O-methylguanosine

Um=2′-O-methyluridine

“-” (hyphen)=3′-5′ phosphodiester linkage sodium salt

“-ps-”=3′-5′ phosphorothioate linkage sodium salt

and wherein L96 has the following formula:

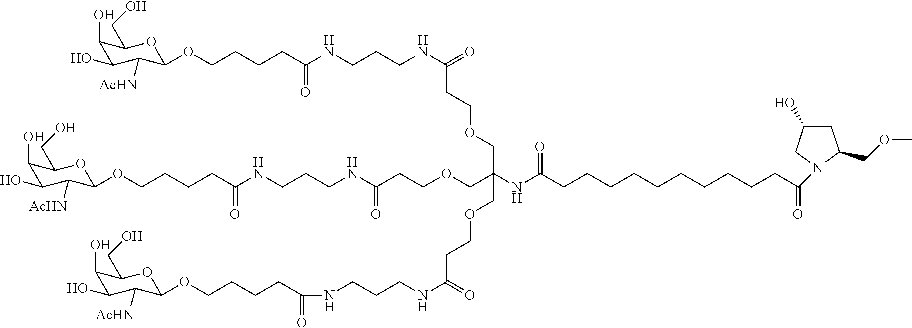 [SMILES: COC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

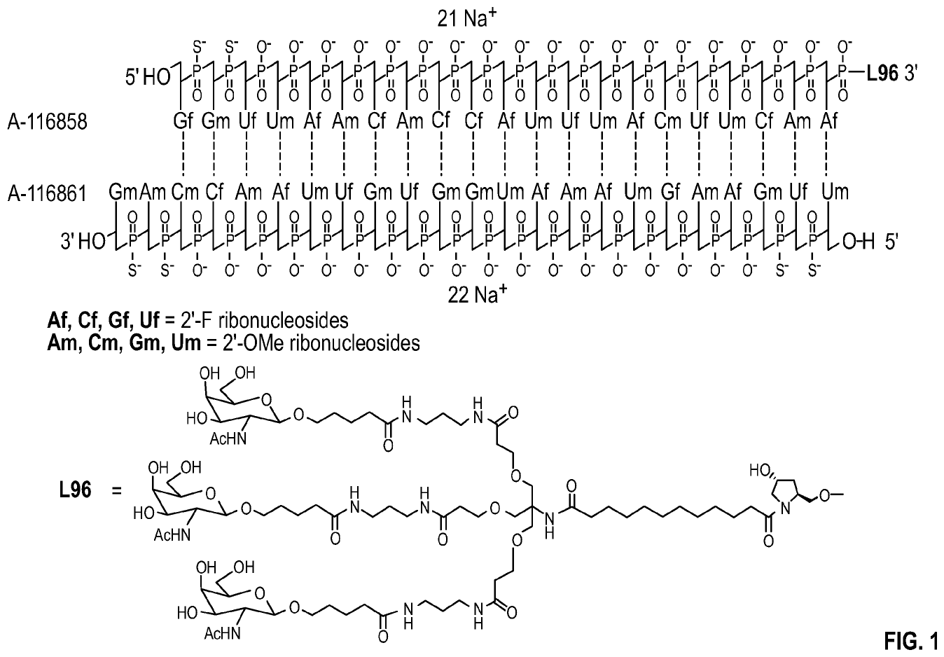

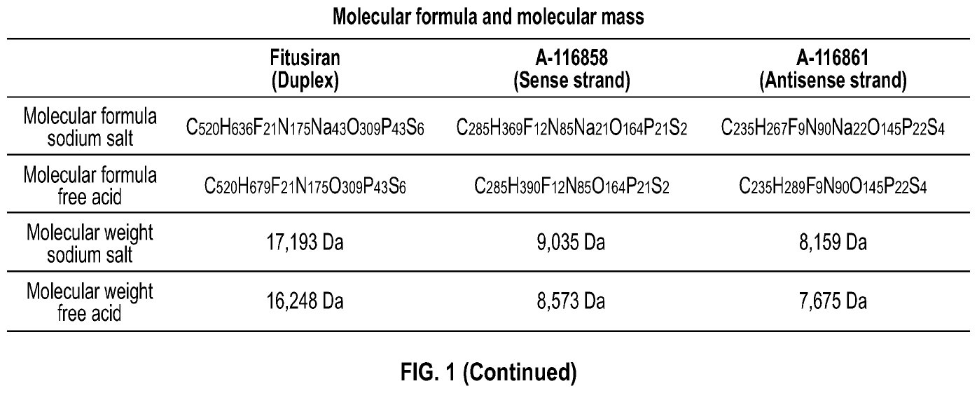

The expanded structural formula, molecular formula, and molecular weight of fitusiran are shown in FIG. 1.

The structure of fitusiran can also be described using the following diagram, wherein the X is O:

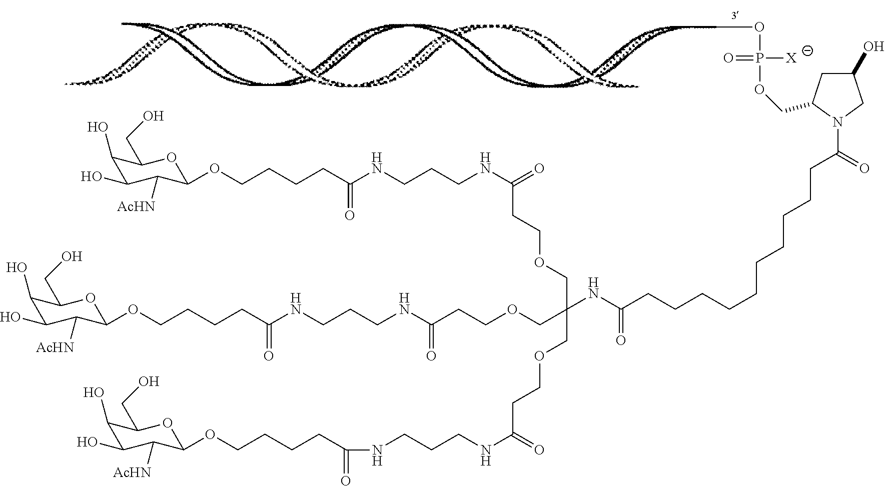

For use in the present treatment methods, fitusiran may be provided in a pharmaceutical composition comprising it and a pharmaceutically acceptable excipient. In certain embodiments, the dsRNA compound is in sodium salt form.

In some embodiments, fitusiran is provided in an aqueous solution at a concentration of 50 to 200 mg/mL (e.g., 50 to 150 mg/mL, 80 to 110 mg/mL, or 90 to 110 mg/mL). As used herein, values intermediate to recited ranges and values are also intended to be part of this disclosure. In addition, ranges of values using a combination of any of recited values as upper and/or lower limits are intended to be included. In further embodiments, the pharmaceutical composition comprises fitusiran at a concentration of 50, 75, 100, 125, 150, or 200 mg/mL.

Unless otherwise indicated, a fitusiran weight recited in the present disclosure is the weight of fitusiran free acid (the active moiety), even though fitusiran is injected to patients subcutaneously in its sodium form (in an aqueous solution). For example, 100 mg/mL fitusiran means 100 mg of fitusiran free acid (equivalent to 106 mg fitusiran sodium, the drug substance) per mL.

In some embodiments, the pharmaceutical compositions comprise fitusiran in a phophate-buffered saline. The phosphate concentration in the solution may be 1 to 10 mM (e.g., 2, 3, 4, 5, 6, 7, 8, or 9 mM), with a pH of 6.0-8.0. The pharmaceutical compositions herein may include a preservative such as EDTA. Alternatively, the pharmaceutical compositions are preservative-free. In particular embodiments, the fitusiran pharmaceutical composition is preservative-free and comprises, consists of, or consists essentially of 100 mg of fitusiran per mL of a 5 mM phosphate buffered saline (PBS) solution. The PBS solution is composed of sodium chloride, dibasic sodium phosphate (heptahydrate), and monobasic sodium phosphate (monohydrate). Sodium hydroxide solution and diluted phosphoric acid may be used to adjust the pH of the composition to 7.0.

The pharmaceutical composition may be provided in a container (e.g., a vial or a syringe). The container may contain single or multiple doses. In some embodiments, the container is a single-use container (e.g., a single-use ampule or a single-use syringe such as a single-use pre-filled syringe), with each container containing 10-100 mg fitusiran (e.g., 10 mg, 20 mg, 25 mg, 40 mg, 50 mg, or 80 mg). The fitusiran may be provided in a solid form in the container and reconstituted in an aqueous solution (e.g., PBS) prior to use, with the reconstituted solution containing 50-150 mg/mL (e.g., 100 mg/mL) fitusiran. In some embodiments, fitusiran is provided in sodium salt form in a single-use glass vial or a single-use prefilled syringe (e.g., one with a safety system). In further embodiments, each vial or syringe contains 80 mg of fitusiran in 0.8 mL (or 50 mg of fitusiran in 0.5 mL, 20 mg of fitusiran in 0.2 mL, or 10 mg of fitusiran in 0.1 mL) of 5 mM phosphate buffered saline solution (pH 7.0); and the solution is administered to patients through subcutaneous injection. The solution can be stored at 2 to 30° C. (e.g., 2 to 8° C.).

In particular embodiments, the fitusiran composition for subcutaneous injection contains fitusiran in a 5 mM phosphate buffered saline having 0.64 mM NaH₂PO₄, 4.36 mM Na₂HPO₄, and 84 mM NaCl at pH 7.0. In certain embodiments, the composition of fitusiran solution for subcutaneous injection is shown in Table 1 below:

TABLE 1

| | Composition | | | |
|---|---|---|---|---|
| | | | | Per unit |
| | | | Per unit | (1 mL |
| | Percentage | Per ml | (2 mL vial) | syringe) |
| Components | [%] | [mg] | [mg] | [mg] |
| Fitusiran (active | 10 | 100 [106] | 80 [84.8] | 80 [84.8] |
| moiety) [equivalent | | | | |
| to fitusiran sodium] | | | | |
| Sodium chloride | 0.49 | 4.909 | 3.927 | 3.927 |
| Dibasic sodium | 0.12 | 1.169 | 0.935 | 0.935 |
| phosphate | | | | |
| (heptahydrate) | | | | |
| Monobasic sodium | <0.01 | 0.0885 | 0.0708 | 0.0708 |
| phosphate | | | | |
| (monohydrate) | | | | |
| Phosphoric acid, | — | q.s. | q.s. | q.s. |
| concentrated | | pH 7.0 | pH 7.0 | pH 7.0 |
| Sodium hydroxide | — | q.s. | q.s. | q.s. |
| | | pH 7.0 | pH 7.0 | pH 7.0 |
| Water for injection | q.s. 100 | q.s. 1 mL | q.s. 0.8 mL | q.s. 0.8 mL |

q.s.: quantum satis.

### II. THERAPEUTIC USE OF FITUSIRAN

Fitusiran can suppress liver production of antithrombin (AT). In its role as an anti-coagulant, AT regulates hemostasis by directly targeting thrombin production or by inactivating uncomplexed FXa, which in turn reduces thrombin production (Quinsey et al., Int J Biochem Cell Biol. (2004) 36(3):386-9). Fitusiran may be used to treat those who have impaired hemostasis. For example, fitusiran can be used to treat patients with hemophilia A or B with or without inhibitors for routine prophylaxis to prevent or reduce the frequency of bleeding episodes. In particular embodiments, fitusiran is used to treat patients, for example adult and adolescent patients (≥12 years of age), with hemophilia A or B (congenital factor VIII or factor IX deficiency) with or without inhibitors.

The present methods include administering to the hemophilia patient (e.g., a hemophilia A or B patient) in need thereof a therapeutically effective amount of fitusiran. “Therapeutically effective amount” refers to the amount of fitusiran that helps the patient to achieve a desired clinical endpoint. A desired clinical endpoint may be, for example, reduction of annual bleeding rates (ABR) to no more than 3, no more than 2, no more than 1, or zero. A desired clinical endpoint may also be, for example, reduction of annual spontaneous bleeding rate (AsBR) to no more than 1, and preferable zero.

The present improved treatment methods are based in part on the discovery that the risk of vascular thrombotic events in patients exposed to fitusiran may increase with lower AT levels. AT measurements can be performed by well-established methods, including both kinetic and chromogenic assays. One commonly used method is the INNOVANCE™ Antithrombin assay (Siemens Healthineers, Malvern, PA; U.S. FDA 510(k) #K081769). INNOVANCE™ is a chromogenic assay that quantifies functionally active AT in human citrated plasma based on the inhibition of an excess of factor Xa by AT. The assay may be performed by using an automated coagulation instrument (e.g., Siemens BCS® XP, Sysmex® CA-600 and CS Systems, or Atellica® COAG 360 System), and may be calibrated using Siemens standard human plasma (SHP) with a defined value of AT activity calibrated against World Health Organization (WHO) reference plasma. An equivalent assay is the Dade Behring Berichrom™ Antithrombin III assay (Dade Behring Marburg GmbH, Marburg, Germany; U.S. FDA 510(k) #K933125). Each measurement may be controlled by two independent controls (low and normal value) also calibrated against the WHO standard. The AT activity (%) in a plasma sample is calculated against the WHO reference plasma. 100% AT level is defined as 1 unit of antithrombin activity in 1 mL of reference plasma sample. The limit of detection of the INNOVANCE™ assay is 6.0% based on the assay's U.S. FDA 510(k) decision summary. AT levels range from about 80% to about 120% in the general population.

It has been observed that the risk of arterial thrombotic events among patients receiving fitusiran may increase with AT levels<10%. To mitigate the risk of vascular thrombotic events while maintaining a favorable benefit-risk balance for patients on fitusiran, a patient, for example an adult patient (≥18 years of age) or an adolescent patient (12 to 17 years of age, inclusive), may start on a fitusiran therapy by subcutaneous injection of 50 mg fitusiran every two months (or every eight weeks). The patient's AT level is monitored periodically (e.g., every one, two, three, four, five, six, seven, or eight weeks, or every one, two, three, four, five, or six months). If the patient has two AT measures of <15% (e.g., <10%), the patient will discontinue fitusiran treatment. In some embodiments, upon the first AT level<15%, the patient has another AT activity level sample drawn within a month (e.g., within one or two weeks). If this result is <15%, this will be considered the second AT<15%. Patients receiving fitusiran at a dose of 50 mg Q2M with more than 1 (e.g., 2) AT activity levels<15% will discontinue fitusiran.

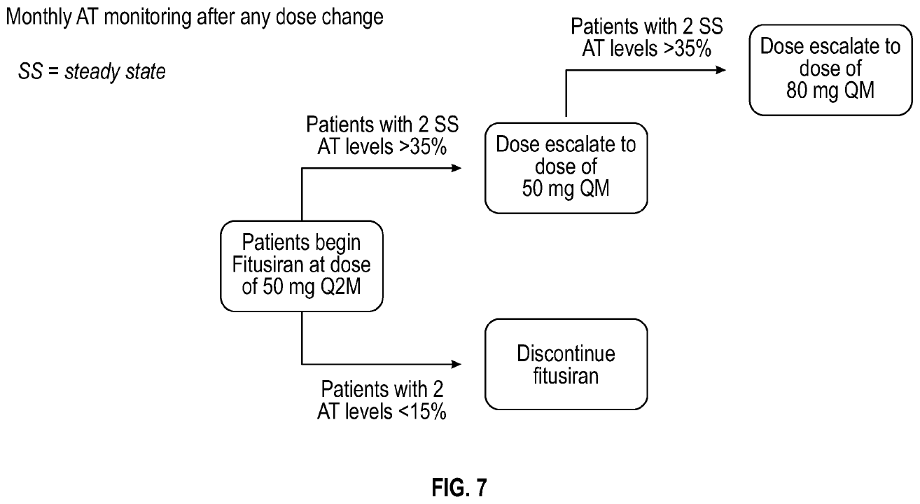

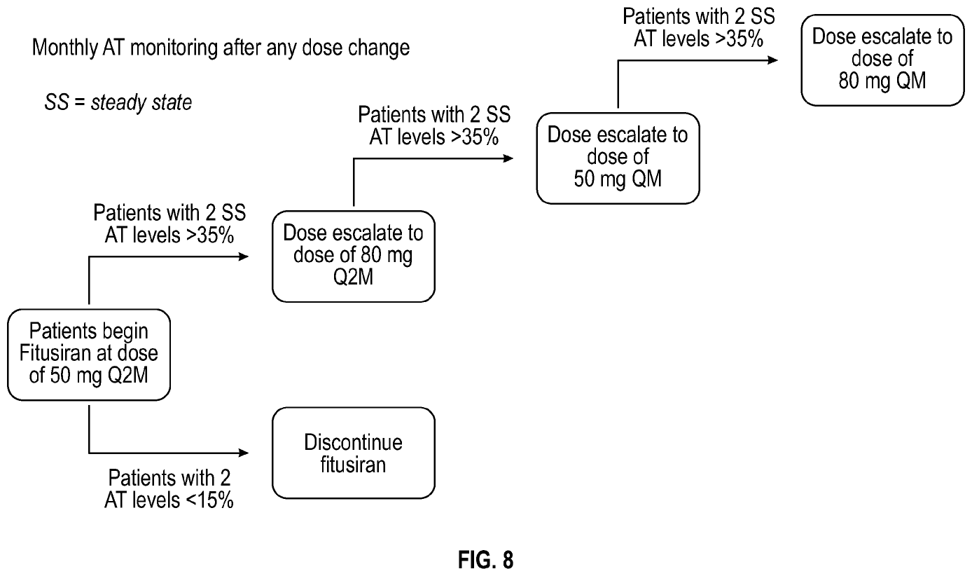

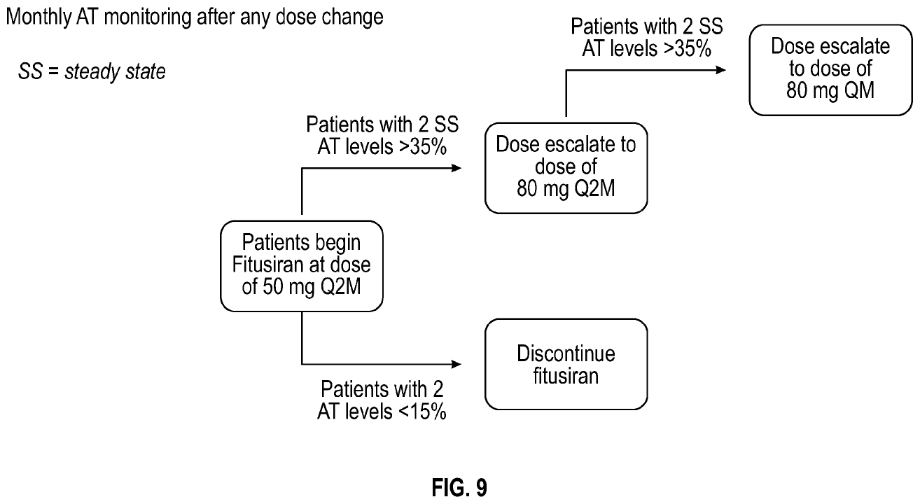

However, if the 50 mg/Q2M (or Q8W) patient has two AT measures of >25% (e.g., >35%), the patient will escalate the dosing regimen as illustrated in FIGS. 7-9. In the approach of FIG. 7, the patient may receive fitusiran at 50 mg every month (or every four weeks); if the patient has two AT measurements of >25% (e.g., >35%) under the 50 mg/QM (Q4W) regimen, the patient may receive fitusiran at 80 mg every month (or every four weeks). In the approach of FIG. 8, the 50 mg/Q2M (or Q8W) patient may receive fitusiran at 80 mg every two months (or every eight weeks); if the patient has two AT measurements of >25% (e.g., 35%) under the 80 mg/Q2M (or Q8W) regimen, the patient may receive fitusiran at 50 mg every month (or every four weeks); if he has two AT measurements of >25% (e.g., 35%) under the 50 mg/QM (or Q4W) regimen, he will receive fitusiran at 80 mg every month (or every four weeks). In the approach of FIG. 9, the 50 mg/Q2M (or Q8W) patient may receive fitusiran at 80 mg every two months (or every eight weeks); if the patient has two AT measurements of >25% (e.g., >35%) under the 80 mg/Q2M (or Q8W) regimen, the patient may receive fitusiran at 80 mg every month (or every four weeks).

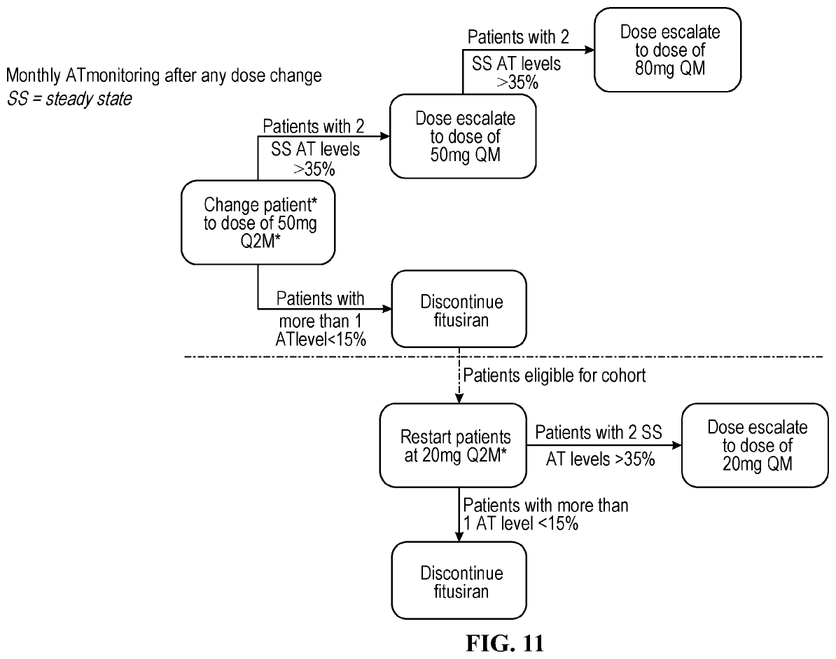

Patients who have discontinued fitusiran after having more than one (e.g., 2) AT activity levels<15% when receiving fitusiran at a dose of 50 mg Q2M may receive fitusiran at a dose of 20 mg Q2M once their AT levels have returned to ≥22% (FIG. 11). Patients receiving fitusiran at a dose of 20 mg Q2M with more than 1 (e.g., 2) AT activity level<15% will discontinue fitusiran treatment. If a patient receiving fitusiran at a dose of 20 mg/Q2M (or Q8W) has two AT measures of >25% (e.g., >35%), the patient may receive fitusiran at 20 mg QM or Q4W.

In the above dose finding regimens, AT measurements for dosing determination are those taken during steady state (SS) of AT activity, i.e., once the patient's AT levels have been stabilized (at low AT activity range) after fitusiran treatment. The SS is typically reached after two or three doses of fitusiran. AT measurements for dosing determination are taken at an appropriate interval (e.g., every four weeks or every eight weeks).

In the above dose finding regimens, the starting dose of 50 mg fitusiran Q2M is included as an illustrative example. For example, a starting dose of fitusiran may be 50 mg Q2M, 20 mg Q2M, 20 mg QM, or 10 mg QM. Dose escalation and de-escalation can then be carried out accordingly from each starting dose. For example, a starting dose of 20 mg Q2M fitusiran can be escalated to 20 mg QM, 50 mg Q2M, 50 mg QM, or 80 mg QM, optionally sequentially in that order, or de-escalated to 10 mg QM.

An AT level of 10-35% (e.g., 10-25%, 15-35%, or 15-25%) is targeted to mitigate the risk of vascular thrombotic events while maintaining a favorable benefit-risk balance for patients on fitusiran. Thus, so long as the patient reaches this targeted AT level, there is no need for the patient to receive a higher fitusiran dosage or more frequent dosing. That is, he remains on the current treatment regimen (i.e., maintenance regimen). For example, once the desired AT level is reached, the patient may be treated with a subcutaneous dose of fitusiran (e.g., 40-90 mg per dose) at an interval of, e.g., every one, two, three, four, five, six, seven, or eight weeks, or every one, two, three, four, five, or six months. In some embodiments, if the patient has two AT measurements of no greater than 35% while receiving 50 mg Q2M, he will maintain this dosing regimen, with no need to further escalate the dosage or dosing frequency. As another example, if the patient has two AT measurements of no greater than 35% while receiving 80 mg Q2M or 50 mg QM, he will remain on this dosing regimen, with no need to further escalate the dosage or dosing frequency (to, e.g., 80 mg QM). However, fitusiran treatment should be discontinued if a patient has more than 1 (e.g., 2) AT measurements<15% (e.g., <10%) as a risk mitigation measure for vascular thrombotic events. Alternatively, the patient may resume treatment with a lower dose of fitusiran after their AT levels have returned to above 15%, e.g., ≥22%.

In some embodiments of maintenance regimens, a patient with hemophilia A or B with or without inhibitors is treated with a subcutaneous dose of fitusiran at 50 mg per dose every two months (or every eight weeks). In other embodiments, a patient with hemophilia A or B with or without inhibitors is treated with a subcutaneous dose of fitusiran at 50 mg every month (or every four weeks). In yet other embodiments, a patient with hemophilia A or B with or without inhibitors is treated with a subcutaneous dose of fitusiran at 80 mg every two months (or every eight weeks). In yet other embodiments, a patient with hemophilia A or B with or without inhibitors is treated with a subcutaneous dose of fitusiran at 80 mg every month (or every four weeks). In yet other embodiments, a patient with hemophilia A or B with or without inhibitors is treated with a subcutaneous dose of fitusiran at 20 mg every two months (or every eight weeks). In yet other embodiments, a patient with hemophilia A or B with or without inhibitors is treated with a subcutaneous dose of fitusiran at 20 mg every month (or every four weeks). In yet other embodiments, a patient with hemophilia A or B with or without inhibitors is treated with a subcutaneous dose of fitusiran at 10 mg every month (or every four weeks).

In some embodiments, patients may receive periodic (e.g., monthly or every four weeks) AT monitoring for 12 months following a change in fitusiran dosing regimen.

In some embodiments, once a patient stays on a maintenance regimen (e.g., 10 mg QM or Q4W, 20 mg Q2M or Q8W, 20 mg QM or Q4W, 50 mg Q2M or Q8W, 50 mg QM or Q4W, 80 mg QM or Q4W, or 80 mg Q2M or Q8W), the patient may receive less frequent AT monitoring. For example, his AT level may be monitored every month, every two months, every three months, every four months, semi-annually, annually, or every two years.

### III. PATIENT MANAGEMENT

Patients on fitusiran are monitored for hemostasis parameters, e.g., coagulation parameters (D-dimer, prothrombin fragment 1+2, and fibrinogen) and for signs and symptoms of vascular thrombotic events. Such signs and symptoms may include, but are not limited to, severe or persistent headache, headache with nausea and vomiting, chest pain and/or tightness, coughing up blood, trouble breathing, abdominal pain, fainting or loss of consciousness, swelling or pain in the arms or legs, vision problems, weakness and/or sensory deficits, and changes in speech. An evaluation of signs and symptoms potentially consistent with vascular thrombosis should include appropriate imaging studies as applicable. For the diagnosis of cerebral venous sinus thrombosis magnetic resonance imaging venogram (MRV) or computed tomography venogram (CTV) is recommended.

If a patient develops thrombosis while on fitusiran, AT reversal may be administered in combination with a replacement factor or BPA and appropriate anticoagulation. AT reversal should follow labeled product recommendations for the prevention of perioperative thrombosis in patients with AT deficiency, and individualize patient doses to target 80-120% AT activity. The use of plasma derived AT may be preferable to recombinant AT, given its longer half-life.

Bleeding events in patients on fitusiran may be managed by on-demand administration of a replacement factor (recombinant or plasma-derived Factor VIII or Factor IX) or a BPA (e.g., fresh-frozen plasma (FFP); rFVIIa; and aPCC). The amount of the factor or BPA must be reduced in patients on fitusiran to prevent vascular thrombosis. See, e.g., WO 2019/014187.

### IV. EXEMPLARY EMBODIMENTS

Further non-limiting exemplary embodiments of the present disclosure are shown below.

1. A method of prophylactic treatment of a patient with hemophilia A or B with or without inhibitors, comprising:

a) subcutaneously administering to the patient in need thereof 50 mg of fitusiran every two months or every eight weeks,

b) obtaining a measurement of an antithrombin (AT) level in the patient, and

c) subcutaneously administering to the patient 80 mg of fitusiran every two months or every eight weeks, or 50 mg of fitusiran every month or every four weeks, if the AT level is >X %, wherein X=25 or 35.

2. A method of reducing thrombotic risk in a patient receiving fitusiran for prophylactic treatment of hemophilia A or B with or without inhibitors, comprising:

a) subcutaneously administering to the patient in need thereof 50 mg of fitusiran every two months or every eight weeks,

b) obtaining a measurement of an antithrombin (AT) level in the patient, and

c) subcutaneously administering to the patient 80 mg of fitusiran every two months or every eight weeks, or 50 mg of fitusiran every month or every four weeks, if the AT level is >X %, wherein X=25 or 35.

3. The method of embodiment 1 or 2, further comprising: d) continuing step a) if the AT level is Y-X %, wherein Y=10 or 15.
4. The method of any one of embodiments 1-3, further comprising: e) discontinuing fitusiran treatment if the AT level is <Y %, wherein Y=10 or 15.
5. The method of any one of embodiments 1-4, wherein step b) is performed at least twice and step c) is performed only when both measured AT levels are >X %, or step e) is performed only when both measured AT levels are <Y %.
6. The method of any one of embodiments 1-5, further comprising, after performing step c):

i) obtaining a measurement of AT level in the patient; and

ii) subcutaneously administering to the patient 80 mg of fitusiran every month or every four weeks, if the AT level of step i) is >25% or >35%, or

iii) otherwise continuing step c).

7. The method of embodiment 6, wherein step i) is performed at least twice and step ii) is performed only when both measured AT levels of step i) are >25% or >35%.
8. The method of any one of embodiments 1-5, further comprising, after performing step c) wherein step c) comprises subcutaneously administering to the patient 80 mg of fitusiran every two months or every eight weeks:

i) obtaining a measurement of AT level in the patient; and

ii) subcutaneously administering to the patient 50 mg of fitusiran every month or every four weeks, if the AT level of step i) is >25% or >35%, or

iii) otherwise continuing step c).

9. The method of embodiment 8, wherein step i) is performed at least twice and step ii) is performed only when both measured AT levels of step ii) are >25% or >35%.
10. The method of embodiment 8 or 9, further comprising, after performing step ii):

A) obtaining a measurement of AT level in the patient; and

B) subcutaneously administering to the patient 80 mg of fitusiran every month or every four weeks, if the AT level of step A) is >25% or >35%, or

C) otherwise continuing step ii).

11. The method of embodiment 10, wherein step A) is performed at least twice and step B) is performed only when both measured AT levels are >25% or >35%.
12. A method of prophylactic treatment of a patient with hemophilia A or B with or without inhibitors, wherein the patient is twelve years of age or older, comprising:

(a) subcutaneously administering to the patient in need thereof fitusiran at a starting dose amount at a selected dosing frequency;

(b) obtaining a measurement of an antithrombin (AT) level in the patient; and

(c) performing one of the following steps:
(i) if the AT level is 15-35%, repeating step (a),
(ii) if the AT level is >35%, subcutaneously administering to the patient fitusiran at a higher dose amount at the selected dosing frequency, or at the starting dose amount at a higher dosing frequency, or
(iii) if the AT level is <15%, discontinuing or pausing fitusiran treatment.
   13. A method of reducing thrombotic risk in a patient receiving fitusiran for prophylactic treatment of hemophilia A or B with or without inhibitors, wherein the patient is twelve years of age or older, comprising:

(a) subcutaneously administering to the patient in need thereof fitusiran at a starting dose amount at a selected dosing frequency;

(b) obtaining a measurement of an antithrombin (AT) level in the patient; and

(c) performing one of the following steps:
(i) if the AT level is 15-35%, repeating step (a),
(ii) if the AT level is >35%, subcutaneously administering to the patient fitusiran at a higher dose amount at the selected dosing frequency, or at the starting dose amount at a higher dosing frequency, or
(iii) if the AT level is <15%, discontinuing or pausing fitusiran treatment.
   14. The method of embodiment 12 or 13, wherein the starting dose amount of fitusiran is selected from 10 mg to 50 mg.
   15. The method of any one of embodiments 12-14, wherein the selected dosing frequency is every two months (Q2M) or every eight weeks (Q8W), optionally wherein the higher dosing frequency is every month (QM) or every four weeks (Q4W).
   16. The method of embodiment 15, wherein the starting dose amount is 20 mg and the selected dosing frequency is Q2M or Q8W.
   17. The method of embodiment 15, wherein the starting dose amount is 50 mg and the selected dosing frequency is Q2M or Q8W, optionally wherein

step (c)(ii) is performed if two measurements of the AT level are >35%, or

step (c)(iii) is performed if more than one measurement, optionally two measurements, of the AT level is <15%.

18. The method of embodiment 17, wherein step (c)(ii) comprises subcutaneously administering to the patient fitusiran at 50 mg QM.
19. The method of embodiment 18, comprising, after step (c)(ii), subcutaneously administering to the patient fitusiran at 80 mg QM if the AT level in the patient after step (c)(ii) is >35%, optionally according to two measurements.
20. The method of any one of embodiments 17-19, comprising, after step (c)(iii):

(A) subcutaneously administering fitusiran to the patient at 20 mg Q2M;

(B) if the AT level of the patient after step (A) is
<15%, optionally according to more than measurement, discontinuing fitusiran treatment,
15-35%, repeating step (A), or
>35%, optionally according to two measurements, subcutaneously administering to the patient fitusiran at 20 mg QM.
   21. The method of any one of the preceding embodiments, further comprising subcutaneously administering fitusiran to the patient at a dose amount and a dosing frequency sufficient to maintain the AT level in the patient at 15-35%, optionally comprising subcutaneously administering fitusiran at:

10 mg QM or Q4W,

20 mg QM or Q4W,

20 mg Q2M or Q8W,

50 mg QM or Q4W,

50 mg Q2M or Q8W, or

80 mg QM or Q4W.

22. The method of any one of embodiments 1-21, wherein AT level measurements are obtained after the patient has received at least two doses of fitusiran in the prior step.
23. A method of prophylactic treatment of a patient with hemophilia A or B with or without inhibitors, comprising subcutaneously administering to the patient in need thereof 50 or 80 mg of fitusiran every two months or every eight weeks, optionally wherein the patient is twelve years of age or older.
24. A method of prophylactic treatment of a patient with hemophilia A or B with or without inhibitors, wherein the patient is twelve years of age or older, comprising subcutaneously administering to the patient in need thereof 20 mg of fitusiran every month or every four weeks, or every two months or every eight weeks.
25. A method of prophylactic treatment of a patient with hemophilia A or B with or without inhibitors, wherein the patient is twelve years of age or older, comprising subcutaneously administering to the patient in need thereof 10 mg of fitusiran every month or every four weeks.
26. The method of any one of embodiments 1-25, comprising obtaining a measurement of AT level in the patient every four weeks, every eight weeks, every month, every two months, every six months, or every 12 months.
27. The method of any one of embodiments 1-26, wherein fitusiran is provided in a phosphate-buffered saline (PBS) at 50-200 mg/mL, optionally 100 mg/mL, and optionally wherein the PBS has a pH of 7.
28. Fitusiran for use in a method of any one of embodiments 1-27.
29. An article of manufacture for use in a method of any one of embodiments 1-27, optionally wherein the article of manufacture is a kit.
30. The article of manufacture for use of embodiment 29, wherein the article of manufacture is a container containing one or more doses of fitusiran, each dose being 80 mg, 50 mg, 20 mg, or 10 mg, optionally wherein

the 80 mg of fitusiran is in 0.8 mL of a phosphate-buffered saline (PBS),

the 50 mg of fitusiran is in 0.5 mL of a PBS,

the 20 mg of fitusiran is in 0.2 mL of a PBS, or

the 10 mg of fitusiran is in 0.1 mL of a PBS, and

optionally wherein the PBS has a pH of 7

31. The article of manufacture for use of embodiment 30, wherein the container is a single-use, single-dose prefilled syringe.
32. Use of fitusiran for the manufacture of a medicament to treat hemophilia in a method of any one of embodiments 1-27.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of hematology, medicine, medicinal and pharmaceutical chemistry, and cell biology described herein are those well-known and commonly used in the art. Further, unless otherwise required by context, singular terms shall include pluralities and plural terms shall include the singular. Throughout this specification and aspects, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain aspects, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this invention may be better understood, the following examples are set forth. These examples are for purposes of illustration only and are not to be construed as limiting the scope of the invention in any manner.

### EXAMPLES

### Example 1: Evaluation of Vascular Thrombotic Events in Fitusiran Treatment

Hemostasis is a highly intricate biological process. To improve the safety of fitusiran treatment of hemophilia A and hemophilia B patients, occurrences and risks of vascular thrombotic events were carefully monitored in patients in clinical trials.

A Phase I clinical study of fitusiran was completed. In one of the arms (“Part B”), 12 male non-inhibitor patients with hemophilia A or B were treated with a weekly regimen of fitusiran ranging from 0.015 mg/kg to 0.075 mg/kg for three weeks. In another arm (“Part C”), 18 male non-inhibitor patients (including five patients who received fitusiran in the aforementioned arm) received a monthly regimen of fitusiran ranging from 0.225 mg/kg to 1.8 mg/kg or a fixed dose of 80 mg, for three months. In yet another arm (“Part D”), 17 male inhibitor patients received a monthly fixed-dose of 50 mg or 80 mg of fitusiran. All doses were administered subcutaneously. Serum samples were collected after administration of fitusiran to monitor AT protein levels, AT activity, and duration of AT protein silencing. AT protein levels were monitored using ELISA and AT activity levels were monitored using a chromogenic assay for the quantification of functionally active AT. Thrombin generation (TG) was measured by generation of thrombin generation curves using a Calibrated Automated Thrombinoscope (tissue factor=1 pM). Fold change in peak thrombin was calculated relative to the average peak thrombin value for two pre-dose values for each subject.

There were no vascular thrombotic events reported in the Phase I study. Any bleed events were successfully managed with standard replacement factor or bypassing agent administration. Furthermore, there were no instances of anti-drug antibody (ADA) formation. The AT knockdown of the patients are described in international publication WO 2017/100236.

Further studies were initiated to evaluate the safety and efficacy of fitusiran in male adult and adolescent patients (≥12 years of age) with moderate or severe hemophilia A or B with or without inhibitors. Reports of vascular (arterial) thrombotic events were received.

### Example 2: Analysis of Association of AT Levels to Thrombotic Events

Based on the mechanism of action of fitusiran and observation of multiple AT measurements less than 10% in patients with reported thrombotic events, AT levels were considered possibly contributory and evaluated as a potential modifiable target for risk mitigation. Specific analyses were conducted, including AT levels in both the fitusiran population and patients with vascular thrombotic events, the incidence rate of vascular thrombotic events by AT level, intra-patient variability in AT measurements, annualized bleeding rate (ABR) by AT level, and simulations to predict AT measurements with various fitusiran dosing regimens.

A. AT Levels in the Fitusiran Population

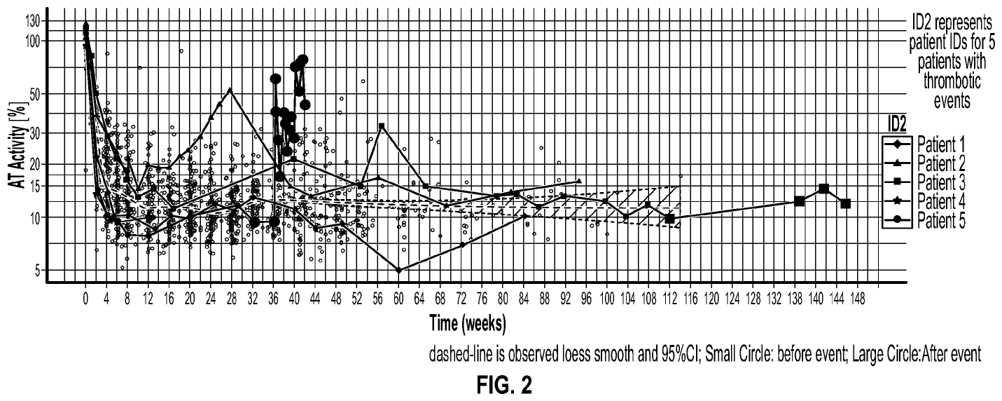

AT activity for the five patients who experienced vascular thrombotic events was compared graphically with AT activity levels of all fitusiran-treated patients in Phase 3 studies. FIG. 2 shows the comparison of AT activity profiles between Phase 3 patients and the five patients with vascular thrombotic events, with actual time in weeks. Only patients who started on fitusiran are included in this graphical analysis. FIG. 2 demonstrated that the AT activities of the five patients with vascular thrombotic events were generally in the range of AT levels of other patients in Phase 3 studies. However, AT levels for the three patients with arterial thrombotic events (Patients 1, 4, and 5) are generally lower than those for the two patients with venous thrombosis (Patients 2 and 3), who used factor or bypassing agent in excess of the revised Bleed Management Guidelines).

B. Incident Rate of Vascular Thrombotic Events by AT Level

The incident rate (IR) of vascular thrombotic events by AT level was determined. For all patients exposed to at least one dose of fitusiran, the total patient years for each of three AT categories was calculated: <10%, 10-20%, and >20%. Several patients had two consecutive AT measurements separated by a long period of time. For these patients, their AT level was assumed to be constant for 90 days following the previous measurement and then missing until the next AT measurement. The five patients with vascular thrombotic events were then included in the AT category representative of their level for the greatest amount of time during fitusiran exposure and an IR per 100 patient years was derived (Table 2; T.E.: thrombotic events).

TABLE 2
Incidence Rate of Vascular Thrombotic
Events by AT Category in Patient Years

| | # of | Total | |
|---|---|---|---|
| AT | patients | Patient | IR of Vascular T.E. |
| Category | with T.E. | Years | per 100 Patient Years |
| <10% | 3 | 50.719 | 5.91 |
| 10-20% | 2 | 134.439 | 1.49 |
| >20% | 0 | 91.803 | 0 |

The IR of vascular thrombotic events in AT categories <10%, 10-20%, and >20% was 5.91, 1.49, and 0 per 100 patient years, respectively. Of note, the patients with vascular thrombotic events included in the AT category <10% represent those with arterial (or suspected arterial) thrombosis (cerebral infarct, cerebrovascular accident, and spinal vascular disorder), whereas those included in the AT category 10-20% represent those with venous events that occurred in the setting of factor or bypassing agent use in excess of the Revised Bleed Management Guidelines (cerebral venous sinus thrombosis and atrial thrombosis).

The etiology of vascular thrombotic events in the fitusiran population was likely multifactorial. However, these data suggest that the risk of vascular thrombotic events may be increased with lower AT levels; specifically, the risk of arterial thrombotic events may be greater with AT levels <10%. A decrease in AT reduction is a potential risk mitigation measure for vascular thrombotic events and may be achieved with changes in dosing regimen (dose and/or frequency).

C. AT Levels and Intra-Individual Variability

To determine an appropriate AT target to minimize levels<10%, intra-individual variability was assessed. The intra-individual variability in AT activity was assessed during steady state. This was to evaluate the intra-patient variability in AT activity once patient's AT levels have been stabilized (at low AT activity range) after fitusiran treatment. To estimate intra-individual variability, AT activity assessments from day 56 to day 196 were used from patients who were on fitusiran treatment. Day 56 was chosen as starting time since with the 80 mg monthly regimen, as patients were expected to reach steady AT activity by day 56. From all the Phase 3 patients who had measured AT activity between day 56 and day 196, patients who had less than four AT activity assessments were excluded. Patients who had significant dose interruptions during the day 56 and day 196 period were also excluded from the intra-individual variability assessment. Additionally, five patients who experienced thrombotic events were included in the steady state intra-individual variability assessment, even if any of these patients had <4 AT observations.

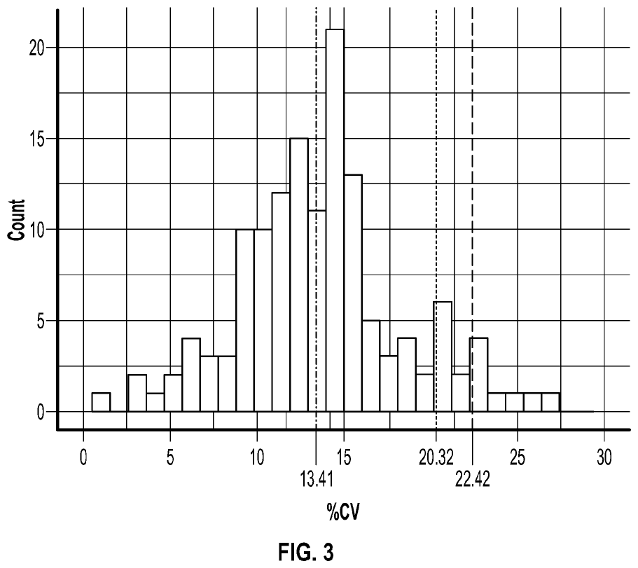

Overall, AT activity measurements from 139 patients out of a total of 259 patients enrolled satisfied the required criteria mentioned above and were used to assess intra-individual variability. For each of the 139 patients, the within patient AT activity mean, standard deviation (SD) and coefficient of variation (% CV) were computed; % CV was computed as SD*100/mean. The distribution of % CV for the eligible patients is shown in FIG. 3. The median, 90th percentile and 95th percentile for the % CV are 13.4%, 20.3% and 22.4%, respectively; represented by the vertical red line, the vertical blue line and the vertical green line respectively.

The 95th percentile for the % CV of 22.4%, when applied to AT activity of 15% and 16% results in a lower bound of 90% confidence interval of 9.5% and 10.1% respectively. This shows that an AT activity level of 15% is a reasonable threshold to minimize the occurrence of AT activity levels<10%.

In addition to the above analysis, the lowest threshold of average AT to achieve a new AT>10% was calculated based on the 80 mg monthly dosing regimen in chosen studies. The between subject and within subject standard deviations were obtained (3.8% and 3.26%, respectively). AT observation at steady state N times was then assumed (N=1, 2, 3 . . . ). The posterior distribution of a new steady state was derived based on AT values at steady state to obtain the lower threshold of average AT to achieve a 95% chance that a new AT is >10%.

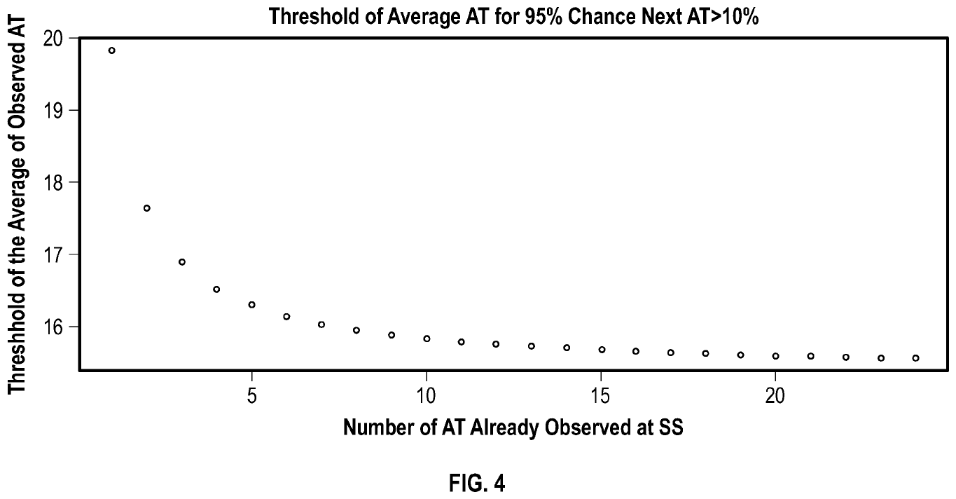

Following 10-15 AT observations, if the average AT level is ˜15%, there is a 95% chance that the next AT level will be >10% (FIG. 4). This analysis further supports the selection of an AT threshold of 15% to minimize the occurrence of AT activity levels<10%.

D. Antithrombin Levels and Efficacy

1. Antithrombin

The coagulation system is a balanced process with the need to maintain hemostasis and to prevent thrombosis. This balance is achieved through a well-described, interconnected cascade consisting of both procoagulant (e.g., FVIII, FIX) and anticoagulant (e.g., AT and Protein C) factors. Antithrombin (formerly called ATIII) is a liver-expressed anticoagulant that is a major endogenous inhibitor of thrombin and other serine proteases such as factors Xa and IXa. Based on the therapeutic hypothesis for fitusiran, an increase in thrombin generation is expected as AT is lowered. It has been shown that the efficacy of fitusiran is related to reducing AT levels to approximately 75% of normal. Therefore, the upper threshold of AT level is analyzed to retain efficacy while minimizing the safety risk.

2. AT Reduction Improves Thrombin Generation in Hemophilia Plasma

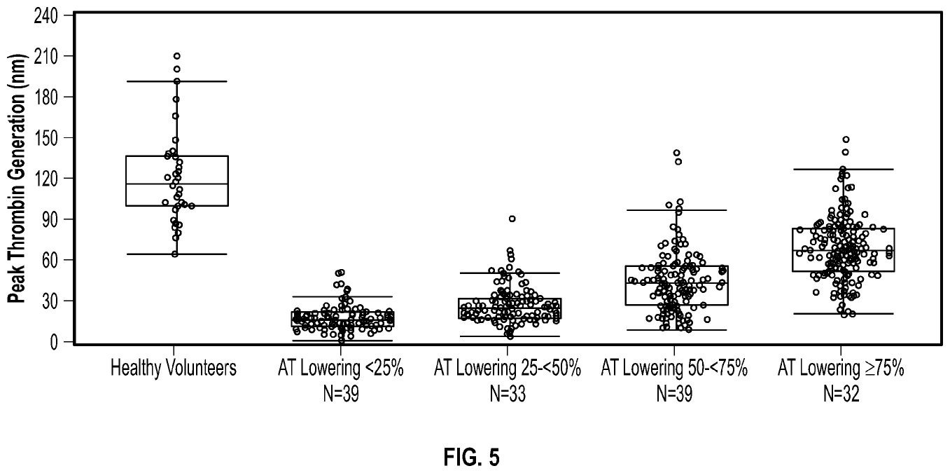

This relationship between AT and thrombin generation (TG) was explored in the Phase I study. In this analysis, all available peak thrombin values were grouped according to their paired AT levels. The peak thrombin values available for patients in Parts B and C grouped by AT lowering quartiles are depicted in FIG. 5. Peak thrombin values for healthy volunteers participating in Part A are also displayed. These data demonstrated an increase in thrombin generation as AT lowering is increased. Of note, median peak thrombin values at the highest quartile of AT lowering (AT lowering >75%) approached the lower end of the range seen in healthy volunteers.

3. AT Level—Bleed Relationship

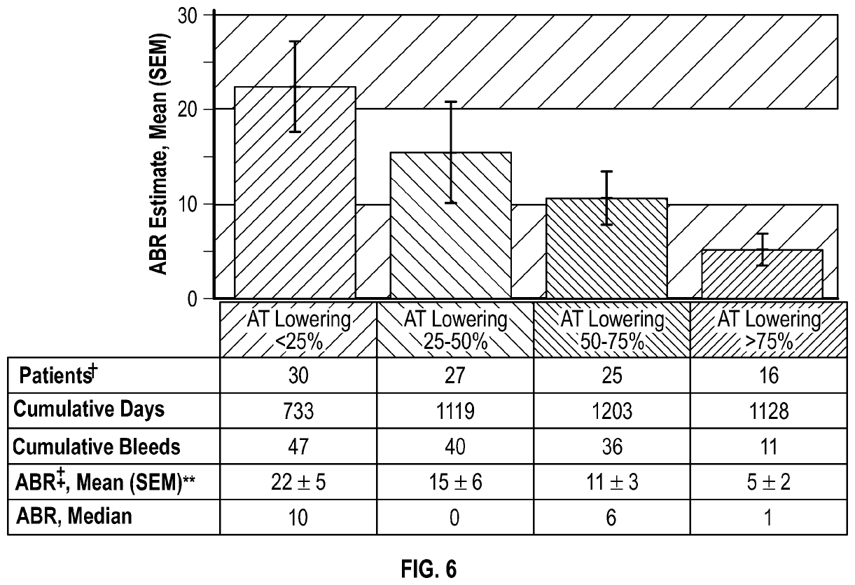

Initial post hoc analysis to determine the relationship of bleeds to AT levels was done utilizing data from a Phase 1 study investigating the safety, tolerability, and pharmacokinetics of fitusiran. Bleed events were grouped by AT quartile for 16 patients and more than 1100 cumulative days with AT lowering >75% (FIG. 5). Based on this early data, there was a clear decrease in bleeding events in patients who had AT lowering >75% with only 11 bleeds in 1128 cumulative days resulting in a median ABR of 1 compared to patients who had less AT lowering (FIG. 6). In summary, when AT is lowered to >75% of normal, TG levels approach the lower end of the healthy volunteer normal range (FIG. 5) and median ABR is observed to be 1 (FIG. 6). Therefore, the optimal therapeutic range of fitusiran has been determined to be when AT levels are >75% of normal or when 25% residual AT levels remain.

To further expand understanding of the relationship of bleeding episodes to AT levels, an ad hoc analysis of available blinded clinical trial treated bleed data was conducted relative to AT levels. The goal of this analysis was to understand the distribution of ABR by AT level above and below 20%. Patients in chosen trials were segregated by AT levels<10%, 10-15%, 15-20% and >20%. Of the 74 patients with AT levels<10% the median ABR was 0.0, in the 116 patients with AT levels between 10% and 15% the median ABR was 1.09, In the 14 patients with AT levels between 15% and 20% the observed median ABR was 0.17 and in the 29 patients with AT levels>20% the median ABR was 1.35 (Table 3).

TABLE 3
Observed ABR by AT Category in Fitusiran Clinical Trials

| AT Category | | | | | | | |
|---|---|---|---|---|---|---|---|
| patients spent | Summary of Individual Observed ABR | | | | | | |
| majority of time | | | | | | | Total |
| while exposed to | | | | | | | Patient |
| fitusiran | n | min | median | max | mean | std | Years |
| <10% | 74 | 0 | 0.00000 | 63.9188 | 2.72981 | 8.31425 | 47.7728 |
| 10-15% | 116 | 0 | 1.09478 | 52.1786 | 5.14452 | 8.55424 | 93.1143 |
| 15-20% | 14 | 0 | 0.16957 | 19.0632 | 4.08359 | 6.83067 | 33.7413 |
| >20% | 29 | 0 | 1.35781 | 35.7113 | 5.42558 | 8.70351 | 65.3470 |

This analysis reinforced the previous data that AT levels<25% had improved efficacy with all subgroups below 20% having an ABR of 1 or less which is similar to therapies currently on the market.

Significant data now exists to provide clarity on the relationship of bleeds to AT level at the lower threshold. However, understanding the upper boundary of this relationship beyond AT level<25% is still a challenge, which has triggering additional modeling.

4. Upper Threshold of AT Activity

Based on relationship between ABR and AT activity lowering in the Phase I study and PK/PD repeated time to event modeling, it was determined that AT lowering of >75% is optimal for efficacy, which translates to approximately AT activity<25% being optimal for efficacy. Furthermore, extrapolating from the analysis to determine the lower threshold of AT activity if the intra-individual variability % CV estimate of 22.4% is assumed, then the upper bound of the 90% confidence interval and of the 95% confidence interval is 34.2% and 35.9% respectively, making the planned upper threshold of 35% AT a reasonable target to expect patients maintain efficacy while accounting for intra-individual variability in AT activity.

E. Dosing Regimen Simulation

Pharmacokinetic/Pharmacodynamic (PK/PD) model that describes the dynamics of plasma AT activity for patients treated with fitusiran was developed and updated on AT activity data from 45 subjects in Phase 1 (ALN-AT3SC-001) and Phase 2 (LTE14762) studies. The parameter estimates of the model are shown in Table 4. The PK/PD model was further validated with phase 3 AT activity data, and validation showed that the model can reasonably capture central tendency and the variability in phase 3 AT activity data. The PK/PD model with the estimated parameters as shown in Table 4 was used to simulate AT activity at steady state for different dosing scenarios in 1000 virtual patients. Each virtual patient is simulated using a unique combination of the PK/PD parameters considering the inter-individual variability. The objective of the simulations was to identify dosing regimen/s that could help majority patients to maintain AT activity between lower threshold (15%) and upper threshold (35%).

TABLE 4
PK/PD Model Parameter Estimates

| | | | | RSE | % CV | |
|---|---|---|---|---|---|---|
| | | | | (relative | (inter- | |
| | | | | standard | individual | |
| | Parameter | Parameter | Parameter | error in | variability; | |
| Parameter | description | unit | estimate | %) | IIV) | Comment |
| K_{A} | absorption rate | 1/h | 0.157 | — | — | FIXED |
| V₂ | liver central | g | 3300 | — | — | FIXED |
| | volume | | | | | |
| V₃ | liver | g | 6075 | — | — | FIXED |
| | peripheral | | | | | |
| | volume | | | | | |
| CL | clearance from | g/h | 39.6 | — | — | FIXED |
| | liver central | | | | | |
| | compartment | | | | | |
| CL₂ | liver inter- | g/h | 27.2 | — | — | FIXED |
| | compartmental | | | | | |
| | clearance | | | | | |
| Q | clearance into | g/h | 0.0195 | — | — | FIXED |
| | RISC | | | | | |
| | compartment | | | | | |
| RV | RISC volume | g | 9375 | — | — | FIXED |
| CLR | clearance from | g/h | 11.31 | 5.15 | 26.72 | Estimated |
| | RISC | | | | | |
| | compartment | | | | | |
| IC₅₀ | RISC | μg/g | 0.0004622 | 16.51 | 51.5 | Estimated |
| | concentration | | | | | |
| | for 50% of | | | | | |
| | maximum | | | | | |
| | effect | | | | | |
| Iₘₐₓ | maximum | — | 0.939 | 1.5 | — | Estimated |
| | inhibition of | | | | | |
| | AT production | | | | | |
| Kᵢₙ | AT production | %/h | 1.23 | 13.8 | — | Estimated |
| | rate | | | | | |
| Kₒᵤₜ | AT | 1/h | 0.0126 | 13.7 | — | Estimated |
| | elimination | | | | | |
| | rate | | | | | |
| F | Dose effect on | — | −0.279 | 29.9 | 8.6 | Estimated |
| | bioavailability | | | | | |

The results of the simulations are shown in Table 5, where Q2M and QM refer to fitusiran dosing every 8 weeks and every 4 weeks respectively; TroughAT and PeakAT are trough and peak AT activities at steady state; and 5th, Median and 95th refer to 5th percentile, median and 95th percentile, respectively.

TABLE 5
Simulated AT Activity Percentiles for Various Doses of Fitusiran

| Regimen | MinAT | TroughAT_5th_SS | TroughAT_Median_SS | TroughAT_95th_SS |
|---|---|---|---|---|
| 50 mg Q2M | 10.5 | 13.9 | 23.3 | 39.7 |
| 50 mg QM | 7.96 | 10.3 | 17.2 | 29.6 |
| 80 mg Q2M | 7.97 | 11.0 | 17.9 | 30.3 |
| 80 mg QM | 6.90 | 8.91 | 13.4 | 22.8 |

| Regimen | PeakAT_5th_SS | PeakAT_Median_SS | PeakAT_95th_SS | MaxAT |
|---|---|---|---|---|
| 50 mg Q2M | 17.87 | 32.37 | 56.42 | 77.5 |
| 50 mg QM | 11.36 | 18.23 | 31.15 | 58.5 |
| 80 mg Q2M | 13.29 | 24.48 | 42.91 | 61.0 |
| 80 mg QM | 9.11 | 14.16 | 23.55 | 41.7 |

The simulation results show that at 80 mg QM regimen the AT activity is generally lower than for 50 mg QM and 50 mg Q2M regimens. There is significant overlap between AT activity range between 80 mg QM (9.11-23.55) and 50 mg QM (11.36-31.15) for peak AT activity and similarly for trough AT activity. For the 50 mg Q2M regimen, due to longer dosing interval, the variation between trough AT activity and peak AT activity is higher. Based on the simulation results, modification of the fitusiran dosing regimen to 50 mg Q2M is expected to achieve AT activity above 10% for all patients.

Based on these simulation results and considering inter-individual variability, with patients on 50 mg Q2M regimen, the peak AT activity at steady state ranges between 17.9% at 5th percentile to 56.4% at 95th percentile (Table 5). Thus, approximately more than 50% of patients will have steady state peak AT activity higher than 25% when dosed with 50 mg Q2M dose. To maintain efficacy in these patients with higher AT activities, a dose increase from 50 mg Q2M to 50 mg QM should be considered. Specifically, after starting with an initial dose of 50 mg Q2M, patients who have two peak AT assessments at steady state>35% would be considered for dose escalation to 50 mg QM. As described above, the threshold of steady state peak AT activity of 35% is chosen to provide some buffer above the efficacious range (<25% AT activity) while accounting for intra-individual variability in AT activity, before considering dose escalation. The peak AT activity is predicted to occur just before the next dose is to be administered, i.e., 2 months or 8 weeks after prior dose of 50 mg Q2M. Patients on 50 mg QM regimen who may have steady state peak AT activity>35% for two consecutive assessments, may need to escalate dose to 80 mg QM regimen.

F. Summary

In summary, the data suggest that the risk of vascular thrombotic events in patients exposed to fitusiran may be increased with lower AT levels; specifically, the risk of arterial thrombotic events may be greater with AT levels<10%. Based on intra-individual variability, a lower AT threshold of 15% has been selected to minimize the occurrence of AT levels<10%.

It has been experimentally determined in the Phase 1 and confirmed in the Phase 3 clinical trials that efficacy of fitusiran resulting in a median ABR of 1 is observed when AT levels are below 25%. Given the intra-individual variability that could mean AT levels at 35% or less. Therefore, patients are expected to have reasonable efficacy up to 35% AT.

Using the PK/PD model describing AT activity on Fitusiran treatment, a starting dose of 50 mg Q2M is chosen to ensure that the majority of patients meet the goal of AT activity>10%. Dose escalation from 50 mg Q2M to 50 mg QM, and subsequently from 50 mg QM to 80 mg QM, would be recommended if patients have two steady state peak AT activities>35%, as described in FIG. 7. Alternatively, dose escalation from 50 mg Q2M to 80 mg Q2M, followed by escalation from 80 mg Q2M to 50 mg QM, and subsequently from 50 mg QM to 80 mg QM, would be recommended if patients have two steady state peak AT activities>35%, as described in FIG. 8. Alternatively, dose escalation from 50 mg Q2M to 80 mg Q2M, and subsequently from 80 mg Q2M to 80 mg QM, would be recommended if patients have two steady state peak AT activities>35%, as described in FIG. 9.

### Example 3: Further Revision of Dosing Guidance Based on Updated PK/PD Model

In a Phase 3 open-label extension study of long-term safety, P t, PD, and exploratory clinical activity of fitusiran, 50 out of 203 (24.6%) patients who received fitusiran at a dose of 50 mg Q2M experienced two AT activity levels<15%, resulting in discontinuation of fitusiran as per study protocol. Subsequently, the PK/PD model was updated. All PK/PD modeling was conducted with the objective of finding dosing regimens that could maintain AT activity between 15% and 35%. The revised model presented in this Example is an update of the model presented in Example 2.

A. Pharmacokinetic/Pharmacodynamic (PK/PD) Modeling Update

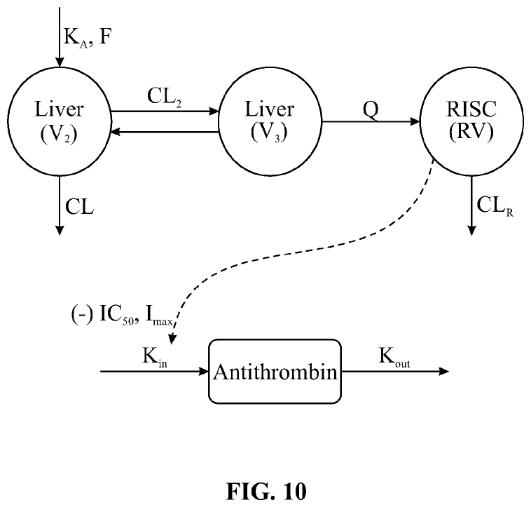

The PK/PD model (FIG. 10) was updated with AT activity data from 274 participants from Phase 1, Phase 2, and Phase 3 studies, with the Phase 3 data including patients treated with fitusiran at a dose of 80 mg QM and 50 mg Q2M.

The parameter estimates of the updated model are shown in Table 6, which updates the parameter estimates presented in Table 4.

TABLE 6
PK/PD Model Parameter Estimates (Updated Model)

| | | | | RSE |
|---|---|---|---|---|
| | | | | (relative |
| | Parameter | Parameter | Population | standard |
| Parameter | description | unit | estimate | error in %) |
| K_{A} | absorption rate | 1/h | 0.157 | — |
| | | | (FIX | |
| V₂ | liver central volume | g | 3300 | — |
| | | | (FIX) | |
| V₃ | liver peripheral volume | g | 6075 | — |
| | | | (FIX) | |
| CL | clearance from liver | g/h | 39.6 | — |
| | central compartment | | (FIX) | |
| CL₂ | liver inter-compartmental | g/h | 27.2 | — |
| | clearance | | (FIX) | |
| Q | clearance into RISC | g/h | 0.0195 | — |
| | compartment | | (FIX) | |
| RV | RISC volume | g | 9375 | — |
| | | | (FIX) | |
| CLR | clearance from RISC | g/h | 11.35 | 2.12 |
| | compartment | | | |
| IC₅₀ | RISC concentration for | μg/g | 0.0002333 | 7.07 |
| | 50% ofmaximum effect | | | |
| Iₘₐₓ | maximum inhibition of | — | 0.9452 | 0.3 |
| | AT production | | | |
| Kᵢₙ | AT production rate | %/h | 1.103 | 4.27 |
| Kₒᵤₜ | AT elimination rate | 1/h | 0.01179 | 4.16 |
| F | Dose effect on | — | −0.27 | — |
| | bioavailability | | (FIX) | |

B. Dosing Regimen Simulations

The updated PK/PD model with the estimated parameters was used to simulate AT activity at steady state for different dosing scenarios in a virtual population of 1000 patients. The dosing regimens simulated were 80 mg QM, 50 mg QM, 50 mg Q2M, 20 mg QM, 20 mg Q2M, and 10 mg QM, with the objective of maintaining AT activity of patients in the range of 15%≤AT≤35%. The results of the simulations are shown in Table 7, which updates the simulated AT activity presented in Table 5.

TABLE 7
Simulated AT Activity (%) For Various Doses of Fitusiran (Updated Model)

| Dose and | | | | |
|---|---|---|---|---|
| Regimen | minAT | TroughAT_5th_SS | TroughAT_Median_SS | TroughAT_95th_SS |
| 80 mg QM | 5.28 | 6.63 | 9.31 | 14.49 |
| 50 mg QM | 5.88 | 7.69 | 11.53 | 19.03 |
| 50 mg Q2M | 6.95 | 9.68 | 15.57 | 26.38 |
| 20 mg QM | 7.63 | 11.42 | 19.28 | 33.37 |
| 20 mg Q2M | 9.76 | 15.71 | 27.15 | 44.65 |
| 10 mg QM | 10.13 | 16.95 | 29.52 | 48.51 |

| Dose and | | | | |
|---|---|---|---|---|
| Regimen | PeakAT_5th_SS | PeakAT_Median_SS | PeakAT_95th_SS | maxAT |
| 80 mg QM | 6.82 | 9.71 | 15.42 | 27.39 |
| 50 mg QM | 7.92 | 12.15 | 20.4 | 36.45 |
| 50 mg Q2M | 11.85 | 21.09 | 38.11 | 64.38 |
| 20 mg QM | 12.01 | 20.59 | 35.49 | 58.92 |
| 20 mg Q2M | 20.25 | 36.66 | 59.24 | 90.15 |
| 10 mg QM | 17.97 | 31.51 | 51.19 | 78.72 |

Q2M and QM refer to fitusiran dosing every 8 weeks and every 4 weeks, respectively; TroughAT and PeakAT are trough and peak AT activities; 5th, median and 95th referring to 5th percentile, median and 95th percentile

Based on the simulation results from Table 7, under the 50 mg Q2M dosing regimen, approximately 45.7% of the virtual participants are expected to have AT activity<15% and therefore may require a de-escalation in dose.

The simulations for lower dosing regimens in 1000 de-novo virtual participants, namely 20 mg Q2M and 20 mg QM, predict that 3.8% and 23.4% of the participants would have AT activity level<15%. Thus, based on the simulations, 20 mg Q2M was considered an appropriate de-escalation regimen for the 45.7% participants who may have AT activity level<15% at 50 mg Q2M. On de-escalation to 20 mg Q2M, approximately 8% of the de-escalated participants may have AT activity level<15% and approximately 7% of the de-escalated participants may have AT activity level>35%, while the rest, 84%-85%, of de-escalated participants are predicted to have AT activity level within the target AT window of 15%-35%.

Additionally, the current data and modeling that took into consideration these data suggest that some participants are very sensitive to fitusiran and have low AT activity levels (<15%) even at the 50 mg Q2M dosing regimen. With the objective to maintain potential fitusiran treatment benefits for these specific participants, while adequately mitigating risk, an additional lower dosing cohort has been implemented for these participants.

The participants who start on 50 mg Q2M, and have AT activity level>35%, will have the option of escalating the dose to 50 mg QM. If 50 mg QM also results in AT activity level>35%, then the participant can escalate to 80 mg QM. Clinical criteria for dose escalation are also included with a goal to achieve adequate efficacy while maintaining AT levels above 15%.

C. Summary

To mitigate the risk of vascular thrombotic events for patients on fitusiran, the clinical trial was revised via modification of the fitusiran dose and regimen to reduce AT level reduction. In particular, to provide an option for patients with AT values<15% on a fitusiran dose of 50 mg Q2M, the Phase 3 clinical trial protocol was amended to introduce a lower dose cohort with a reduced dose of 20 mg Q2M, with possible escalation to 20 mg QM (FIG. 11). On a dose of 20 mg Q2M, patients with more than one AT value<15% within a 12-month period are required to discontinue fitusiran. Patients with two steady state AT levels>35% may escalate to a dose of 20 mg QM.

The newly proposed dosing scheme is based on an updated PK/PD model and introduces a reduced dose of fitusiran. Overall, the dosing strategy is still designed to achieve AT activity levels between 15% and 35% and therefore aims to maintain a favorable benefit-risk balance for patients on fitusiran.

1. A method of prophylactic treatment of a patient with hemophilia A or B with or without inhibitors, comprising:
(a) subcutaneously administering to the patient in need thereof fitusiran at a starting dose amount at a selected dosing frequency;
(b) obtaining a measurement of an antithrombin (AT) level in the patient; and
(c) performing one of the following steps:
(i) if the AT level is 15-35%, repeating step (a),
(ii) if the AT level is >35%, subcutaneously administering to the patient fitusiran at a higher dose amount at the selected dosing frequency, or at the starting dose amount at a higher dosing frequency, or
(iii) if the AT level is <15%, discontinuing or pausing fitusiran treatment.

2. A method of reducing thrombotic risk in a patient receiving fitusiran for prophylactic treatment of hemophilia A or B with or without inhibitors, comprising:
(a) subcutaneously administering to the patient in need thereof fitusiran at a starting dose amount at a selected dosing frequency;
(b) obtaining a measurement of an antithrombin (AT) level in the patient; and
(c) performing one of the following steps:
(i) if the AT level is 15-35%, repeating step (a),
(ii) if the AT level is >35%, subcutaneously administering to the patient fitusiran at a higher dose amount at the selected dosing frequency, or at the starting dose amount at a higher dosing frequency, or
(iii) if the AT level is <15%, discontinuing or pausing fitusiran treatment.

3. The method of claim 1, wherein the starting dose amount of fitusiran is selected from 10 mg to 50 mg.

4. The method of claim 1, wherein the selected dosing frequency is every two months (Q2M) or every eight weeks (Q8W), optionally wherein the higher dosing frequency is every month (QM) or every four weeks (Q4W).

5. The method of claim 4, wherein the starting dose amount is 20 mg and the selected dosing frequency is Q2M or Q8W.

6. The method of claim 4, wherein the starting dose amount is 50 mg and the selected dosing frequency is Q2M or Q8W, optionally wherein
step (c)(ii) is performed if two measurements of the AT level are >35%, or
step (c)(iii) is performed if more than one measurement, optionally two measurements, of the AT level is <15%.

7. The method of claim 6, wherein step (c)(ii) comprises subcutaneously administering to the patient fitusiran at 50 mg QM.

8. The method of claim 7, comprising, after step (c)(ii), subcutaneously administering to the patient fitusiran at 80 mg QM if the AT level in the patient after step (c)(ii) is >35%, optionally according to two measurements.

9. The method of claim 6, comprising, after step (c)(iii):
(A) subcutaneously administering fitusiran to the patient at 20 mg Q2M;
(B) if the AT level of the patient after step (A) is
<15%, optionally according to more than measurement, discontinuing fitusiran treatment,
15-35%, repeating step (A), or
>35%, optionally according to two measurements, subcutaneously administering to the patient fitusiran at 20 mg QM.

10. The method of claim 1, further comprising subcutaneously administering fitusiran to the patient at a dose amount and a dosing frequency sufficient to maintain the AT level in the patient at 15-35%, optionally comprising subcutaneously administering fitusiran at:
10 mg QM or Q4W,
20 mg QM or Q4W,
20 mg Q2M or Q8W,
50 mg QM or Q4W,
50 mg Q2M or Q8W, or
80 mg QM or Q4W.

11. The method of claim 1, wherein each AT level measurement is obtained after the patient has received at least two doses of fitusiran at a given dose amount.

12. A method of prophylactic treatment of a patient with hemophilia A or B with or without inhibitors, comprising subcutaneously administering to the patient in need thereof 50 or 80 mg of fitusiran every two months or every eight weeks.

13. A method of prophylactic treatment of a patient with hemophilia A or B with or without inhibitors, comprising subcutaneously administering to the patient in need thereof 20 mg of fitusiran every month or every four weeks, or every two months or every eight weeks.

14. A method of prophylactic treatment of a patient with hemophilia A or B with or without inhibitors, comprising subcutaneously administering to the patient in need thereof 10 mg of fitusiran every month or every four weeks.

15. The method of claim 1, comprising obtaining a measurement of AT level in the patient every four weeks, every eight weeks, every month, every two months, every four months, every six months, or every 12 months.

16. The method of claim 1, wherein the patient is twelve years of age or older.

17. The method of claim 1, wherein fitusiran is provided in a phosphate-buffered saline (PBS) at 50-200 mg/mL, optionally 100 mg/mL, and optionally wherein the PBS has a pH of 7.

18-19. (canceled)

20. An article of manufacture, wherein the article of manufacture is a container containing one or more doses of fitusiran, each dose being 80 mg, 50 mg, 20 mg, or 10 mg,
optionally wherein
the 80 mg of fitusiran is in 0.8 mL of a phosphate-buffered saline (PBS),
the 50 mg of fitusiran is in 0.5 mL of a PBS,
the 20 mg of fitusiran is in 0.2 mL of a PBS, or
the 10 mg of fitusiran is in 0.1 mL of a PBS, and
optionally wherein the PBS has a pH of 7.

21. The article of manufacture of claim 20, wherein the container is a single-use, single-dose prefilled syringe.

22. (canceled)


SEQUENCE LISTING (2 sequences)

SEQ ID NO: 1 - RNA, 21 nt, Artificial Sequence
  source: /note=“Description of Artificial Sequence Synthetic oligonucleotide”
  modified_base (1)..(1): 2′-fluoroguanosine
  misc_feature (1)..(2): /note=“phosphorothioate linkage”
  modified_base (2)..(2): 2′-O-methylguanosine
  misc_feature (2)..(3): /note=“phosphorothioate linkage”
  modified_base (3)..(3): 2′-fluorouridine
  modified_base (4)..(4): 2′-O-methyluridine
  modified_base (5)..(5): 2′-fluoroadenosine
  modified_base (6)..(6): 2′-O-methyladenosine
  modified_base (7)..(7): 2′-fluorocytidine
  modified_base (8)..(8): 2′-O-methyladenosine
  modified_base (9)..(9): 2′-fluorocytidine
  modified_base (10)..(10): 2′-fluorocytidine
  modified_base (11)..(11): 2′-fluoroadenosine
  modified_base (12)..(12): 2′-O-methyluridine
  modified_base (13)..(13): 2′-fluorouridine
  modified_base (14)..(14): 2′-O-methyluridine
  modified_base (15)..(15): 2′-fluoroadenosine
  modified_base (16)..(16): 2′-O-methylcytidine
  modified_base (17)..(17): 2′-fluorouridine
  modified_base (18)..(18): 2′-O-methyluridine
  modified_base (19)..(19): 2′-fluorocytidine
  modified_base (20)..(20): 2′-O-methyladenosine
  modified_base (21)..(21): 2′-fluoroadenosine
  source: /note=“3′ L96 GalNAc containing moiety”
gguuaacaccauuuacuucaa

SEQ ID NO: 2 - RNA, 23 nt, Artificial Sequence
  source: /note=“Description of Artificial Sequence Synthetic oligonucleotide”
  modified_base (1)..(1): 2′-O-methyluridine
  misc_feature (1)..(2): /note=“phosphorothioate linkage”
  modified_base (2)..(2): 2′-fluorouridine
  misc_feature (2)..(3): /note=“phosphorothioate linkage”
  modified_base (3)..(3): 2′-O-methylguanosine
  modified_base (4)..(4): 2′-fluoroadenosine
  modified_base (5)..(5): 2′-O-methyladenosine
  modified_base (6)..(6): 2′-fluoroguanosine
  modified_base (7)..(7): 2′-O-methyluridine
  modified_base (8)..(8): 2′-fluoroadenosine
  modified_base (9)..(9): 2′-O-methyladenosine
  modified_base (10)..(10): 2′-fluoroadenosine
  modified_base (11)..(11): 2′-O-methyluridine
  modified_base (12)..(12): 2′-O-methylguanosine
  modified_base (13)..(13): 2′-O-methylguanosine
  modified_base (14)..(14): 2′-fluorouridine
  modified_base (15)..(15): 2′-O-methylguanosine
  modified_base (16)..(16): 2′-fluorouridine
  modified_base (17)..(17): 2′-O-methyluridine
  modified_base (18)..(18): 2′-fluoroadenosine
  modified_base (19)..(19): 2′-O-methyladenosine
  modified_base (20)..(20): 2′-fluorocytidine
  modified_base (21)..(21): 2′-O-methylcytidine
  misc_feature (21)..(22): /note=“phosphorothioate linkage”
  modified_base (22)..(22): 2′-O-methyladenosine
  misc_feature (22)..(23): /note=“phosphorothioate linkage”
  modified_base (23)..(23): 2′-O-methylguanosine
uugaaguaaaugguguuaaccag